(19) United States
(12) Patent Application Publication
(10) Pub. No.: US 2024/0247049 A1
(43) Pub. Date: Jul. 25, 2024

(54) AGENT FOR RESTORING VISUAL FUNCTION OR AGENT FOR PREVENTING DETERIORATION IN VISUAL FUNCTION

(71) Applicants: KEIO UNIVERSITY, Tokyo (JP); NAGOYA INSTITUTE OF TECHNOLOGY, Nagoya-shi, Aichi (JP)

(72) Inventors: Toshihide Kurihara, Tokyo (JP); Yusaku Katada, Tokyo (JP); Hiromitsu Kunimi, Tokyo (JP); Kazuo Tsubota, Tokyo (JP); Hideki Kandori, Nagoya-shi, Aichi (JP)

(21) Appl. No.: 18/443,512

(22) Filed: Feb. 16, 2024

Related U.S. Application Data
(63) Continuation of application No. 16/329,631, filed on Feb. 28, 2019, now Pat. No. 11,932,679.

(30) Foreign Application Priority Data
Sep. 2, 2016 (JP) 2016172149

(51) Int. Cl. C07K 14/72; A61K 31/7088; A61K 35/76; A61K 38/00; A61K 48/00; A61P 27/02; C07K 14/195; C07K 14/705; C07K 19/00; C12N 15/09; C12N 15/86

(52) U.S. Cl. CPC C07K 14/723; A61K 31/7088; A61K 35/76; A61K 38/00; A61K 48/00; A61P 27/02; C07K 14/195; C12N 15/09; C12N 15/86; C07K 14/705; C07K 19/00; C12N 2750/14143

(57) ABSTRACT

The purpose of the present invention is to provide an agent for restoring a visual function or an agent for preventing the deterioration in a visual function, which has an excellent visual function restoring ability. The agent for regenerating a visual function or the agent for preventing the deterioration in a visual function according to the present invention contains, as an active ingredient, a chimeric protein having both an amino acid sequence for a microorganism-origin ion-transporting receptor rhodopsin and an amino acid sequence for an animal-origin G-protein-coupled receptor rhodopsin. The chimeric protein is preferably one in which an amino acid sequence for a cytoplasm-side second loop and/or a cytoplasm-side third loop in the amino acid sequence for the microorganism-origin ion-transporting receptor rhodopsin is substituted by an amino acid sequence for a cytoplasm-side second loop and/or a cytoplasm-side third loop in the G-protein-coupled receptor rhodopsin.

### TECHNICAL FIELD

The present invention relates to an agent for restoring visual function or agent for preventing deterioration in visual function.

### BACKGROUND ART

Rhodopsin is a photosensitive receptor with a seven transmembrane structure in the retina of humans and animals. Ion channel and ion pump type rhodopsins derived from microorganisms are also known.

For example, Non Patent Literature 1 discloses an ion channel type rhodopsin, channelrhodopsin 2 (ChR2). Further, Non Patent Literature 2 has reported that a certain visual function is restored in mice/rats by introducing a mutant channelrhodopsin into retinal ganglion cells.

### CITATION LIST

### Non Patent Literature

[NPL 1] Bi et al., “Ectopic expression of a microbial-type rhodopsin restores visual responses in mice with photoreceptor degeneration.” Neuron. 2006; 50(1): 23-33
[NPL 2] Tomita et al., “Restoration of the Majority of the Visual Spectrum by Using Modified Volvox Channelrhodopsin-1”, Molecular Therapy (2014); 22 8, 1434-1440

### SUMMARY OF INVENTION

### Technical Problem

However, the effect of restoring visual function of ion channel type rhodopsins is still not considered sufficient, such that there is room for improvement.

The present invention has been conceived in view of the above circumstances. The objective of the invention is to provide an agent for restoring visual function or agent for preventing deterioration in visual function with an excellent capability to restore visual function.

### Solution to Problem

The inventors have found that a chimeric protein prepared by fusing two completely different rhodopsins, i.e., a microorganism derived ion transport rhodopsin and an animal derived G protein-coupled receptor rhodopsin in fact has an excellent capability to restore visual function to complete the present invention. More specifically, the present inventions are composed of the following configurations.
(1) An agent for restoring visual function or agent for preventing deterioration in visual function comprising, as an active ingredient, a chimeric protein having an amino acid sequence of a microorganism derived ion transport receptor rhodopsin and an amino acid sequence of an animal derived G protein-coupled receptor rhodopsin.
(2) The agent for restoring visual function or agent for preventing deterioration in visual function of (1), wherein the chimeric protein has an amino acid sequence of a second loop on a cytoplasm side and/or a third loop on a cytoplasm side of the amino acid sequence of the microorganism derived ion transport receptor rhodopsin, replaced with an amino acid sequence of a second loop on a cytoplasm side and/or a third loop on a cytoplasm side of the G protein-coupled receptor rhodopsin.
(3) The agent for restoring visual function or agent for preventing deterioration in visual function of (1) or (2), wherein the microorganism derived ion transport receptor rhodopsin is a rhodopsin derived from a microorganism of the Gleobacter genus, and the G protein-coupled receptor rhodopsin is a bovine or human derived rhodopsin.
(4) The agent for restoring visual function or agent for preventing deterioration in visual function of any one of (1) to (3), wherein the chimeric protein has an amino acid sequence encoded by a DNA of any one of the following (a) to (d):
(a) a DNA having a base sequence encoding the amino acid sequence of any one of SEQ ID NOs: 1 to 4;
(b) a DNA having a base sequence that can hybridize under a stringent condition with a base sequence complementary to a base sequence encoding the amino acid sequence of any one of SEQ ID NOs: 1 to 4;
(c) a DNA having a base sequence encoding the amino acid sequence of any one of SEQ ID NOs: 1 to 4 with one or more amino acid substitutions, deletions, and/or additions, and having a visual function restoring capability or visual function deterioration preventing capability; and
(d) a DNA consisting of a base sequence encoding an amino acid sequence having 90% or greater homology with the amino acid sequence of any one of SEQ ID NOs: 1 to 4 and having a visual function restoring capability or visual function deterioration preventing capability.
(5) An agent for restoring visual function or agent for preventing deterioration in visual function comprising, as an active ingredient, an expression vector into which a DNA encoding the amino acid sequence of the chimeric protein of any one of (1) to (4) is incorporated.
(6) The agent for restoring visual function or agent for preventing deterioration in visual function of any one of (1) to (5) for use in treating or preventing retinitis pigmentosa.
(7) An adeno-associated virus (AAV) vector or lentivirus vector, to which a sequence of a chimeric protein having an amino acid sequence of a microorganism derived ion transport receptor rhodopsin and an amino acid sequence of an animal derived G protein-coupled receptor rhodopsin is inserted.
(8) Use of an adeno-associated virus (AAV) vector or lentivirus vector, to which a sequence of a chimeric protein having an amino acid sequence of a microorganism derived ion transport receptor rhodopsin and an amino acid sequence of an animal derived G protein-coupled receptor rhodopsin is inserted, for the manufacture of a medicament for restoring visual function or preventing deterioration in visual function.

### Advantageous Effects of Invention

The present invention can attain an excellent capability to restore visual function.

### BRIEF DESCRIPTION OF DRAWINGS

FIG. 1 is an image of a retina of a wild-type mouse injected with AAV2-CAGGS-EGFP-WPRE-pA into the vitreous body observed under a fluorescence microscope.

FIG. 2 is (a) a graph of a result of recording extracellular potential of retinal ganglion cells by a multielectrode array (MEA) for a control retinitis pigmentosa model (rd1) mouse, and (b) a graph of a result of recording extracellular potential of retinal ganglion cells by MEA for a retinitis pigmentosa model (rd1) mouse injected with AAV2-CAGGS-GR/BvRh-WPRE-pA.

FIG. 3 is a diagram showing a result of recording extracellular potential of retinal ganglion cells by a multielectrode array (MEA) for (a) a control retinitis pigmentosa model (rd1) mouse and (b) a retinitis pigmentosa model (rd1) mouse injected with AAV2-CAGGS-GR/BvRh-WPRE-pA. The top row of FIG. 3 displays a raster plot for 10 firings of retinal ganglion cells, and the bottom row of FIG. 3 is a histogram representing the frequency of firings per second on the vertical axis.

### DESCRIPTION OF EMBODIMENTS

While the specific embodiments of the invention are described in detailed hereinafter, the present invention is not limited in any manner to the following embodiments. The present invention can be practiced by applying an appropriate modification within the scope of the objects of the invention. Explanation is omitted when appropriate for portions where a description would be redundant, but such an omission does not limit the gist of the invention.

### <Agent for Restoring Visual Function or Agent for Preventing Deterioration in Visual Function>

The agent for restoring visual function or agent for preventing deterioration in visual function of the invention comprises, as an active ingredient, a chimeric protein having an amino acid sequence of a microorganism derived ion transport receptor rhodopsin and an amino acid sequence of an animal derived G protein-coupled receptor rhodopsin.

Rhodopsin has a pigment called retinal inside, which is activated by receiving light to transmit a visual signal to the brain. Microorganism derived ion transport receptor rhodopsins can be repeatedly activated by absorbing light because they do not release retinal by light irradiation, but are unable to activate a G protein as in animal derived G protein-coupled receptor rhodopsins. Meanwhile, according to the present invention, high activity through the endogenous G protein due to the G protein-coupled receptor rhodopsin while retaining the function of repeated activation of the microorganism derived ion transport receptor/ion channel type receptor rhodopsin can be attained by fusing an animal derived G protein-coupled receptor rhodopsin to a microorganism derived ion transport receptor rhodopsin that can be repeatedly used. Such a fusion rhodopsin is expected to attain an excellent visual restoring effect. In this manner, microorganism derived rhodopsins and animal derived G protein-coupled receptors are receptors with completely different functions. The inventors have actually found that a chimeric protein combining two such receptors has an excellent capability to restore a visual function. Since such a chimeric protein can be repeatedly activated while having high activity as discussed above, an effect of preventing the deterioration in visual function (e.g., suppressing the progression of retinal diseases such as retinitis pigmentosa) is also expected.

Examples of ion transport receptor rhodopsins include ion pump type receptor rhodopsins and ion channel type receptor rhodopsins.

The chimeric protein of the invention is a chimeric protein of a microorganism derived ion transport receptor rhodopsin and a G protein-coupled receptor rhodopsin, having a seven transmembrane structure. It is preferable in the present invention that a chimeric protein of a microorganism derived ion transport receptor rhodopsin and a G protein-coupled receptor rhodopsin is designed to have both high level of function for repeatedly activating the microorganism derived ion transport receptor rhodopsin and G protein activity due to the G protein-coupled receptor rhodopsin. From this viewpoint, it is preferable that the chimeric protein of the invention has an amino acid sequence of a second loop on a cytoplasm side and/or a third loop on a cytoplasm side of the amino acid sequence of the microorganism derived ion transport receptor rhodopsin substituted with an amino acid sequence of a second loop on a cytoplasm side and/or a third loop on a cytoplasm side of the G protein-coupled receptor rhodopsin, because both activities are maintained high and especially because high visual function restoring capability is attained. The “second loop on a cytoplasm side” and “third loop on a cytoplasm side” refer to loops at position 2 from the N-terminus side and position 3 from the N-terminus side among the seven loops, respectively.

Examples of microorganism derived ion transport receptor rhodopsins include rhodopsins derived from microorganisms, e.g., belonging to eubacteria such as the Gleobacter genus and the like, eukaryotes such as the Volvox genus, Chlamydomonas genus, Guillardia genus, and the like. Examples of the Gloeobacter genus include Gloeobacter violaceus and the like. Examples of the Volvox genus include Volvox carteri and the like. Examples of the Chlamydomonas genus include Chlamydomonas reinhardtii and the like. Examples of Guillardia genus include Guillardia theta and the like. Conformational compatibility with a G protein activation loop and the membrane translocation efficiency are considered important for attaining a higher visual restoration/prophylactic effect. Microorganism derived ion transport receptor rhodopsins are thus preferably of the Gloeobacter genus due to the especially excellent conformational compatibility with a G protein activation loop and membrane translocation efficiency. Gloeobacter violaceus is especially preferable among microorganisms of the Gloeobacter genus. It is also preferable to combine and fuse those of a microorganism of the Gloeobacter genus with a bovine or human derived G protein-coupled receptor rhodopsin among animal derived G protein-coupled receptor rhodopsins. The Gloeobacter genus is also preferable in terms of having an important property of being expressed well in E. coli, which is a eubacterium, and human cells, which are eukaryotes.

Examples of animal derived G protein-coupled receptor rhodopsins include rhodopsins derived from a cow, human, mouse, rat, cat, dog, swine, sheep, horse, or the like. Among them, bovine and human derived rhodopsins are particularly preferable.

More specifically, a chimeric protein preferably has an amino acid sequence encoding the DNA of any one of the following (a) to (d):
(a) a DNA having a base sequence encoding the amino acid sequence of any one of SEQ ID NOs: 1 to 4;
(b) a DNA having a base sequence that can hybridize under a stringent condition with a base sequence complementary to a base sequence encoding the amino acid sequence of any one of SEQ ID NOs: 1 to 4;
(c) a DNA having a base sequence encoding the amino acid sequence of any one of SEQ ID NOs: 1 to 4 with one or more amino acid substitutions, deletions, and/or additions, and having a visual function restoring capability or visual function deterioration preventing capability; and
(d) a DNA consisting of a base sequence encoding an amino acid sequence having 90% or greater homology with the amino acid sequence of any one of SEQ ID NOs: 1 to 4 and having a visual function restoring capability or visual function deterioration preventing capability.

The second loop on the cytoplasm side of the G protein-coupled receptor rhodopsin discussed above preferably has an amino acid encoding the DNA of the following (e) to (h):
(e) a DNA having a base sequence encoding the amino acid sequence of SEQ ID NO: 5 or 6;
(f) a DNA having a base sequence that can hybridize under a stringent condition with a base sequence complementary to a base sequence encoding the amino acid sequence of SEQ ID NO: 5 or 6;
(g) a DNA having a base sequence encoding the amino acid sequence of SEQ ID NO: 5 or 6 with one or more amino acid substitutions, deletions, and/or additions; and
(h) a DNA consisting of a base sequence encoding an amino acid sequence having 90% or greater homology with the amino acid sequence of SEQ ID NO: 5 or 6.

The third loop on the cytoplasm side of the G protein-coupled receptor rhodopsin discussed above preferably has an amino acid encoding the DNA of the following (i) to (1):
(i) a DNA having a base sequence encoding the amino acid sequence of SEQ ID NO: 7;
(j) a DNA having a base sequence that can hybridize under a stringent condition with a base sequence complementary to a base sequence encoding the amino acid sequence of SEQ ID NO: 7;
(k) a DNA having a base sequence encoding the amino acid sequence of SEQ ID NO: 7 with one or more amino acid substitutions, deletions, and/or additions; and
(l) a DNA consisting of a base sequence encoding an amino acid sequence having 90% or greater homology with the amino acid sequence of SEQ ID NO: 7.

A base sequence encoding the amino acid sequence of any one of SEQ ID NOs: 1 to 4 is a preferred sequence of a base sequence encoding the chimeric protein of the invention. The base sequence encoding the amino acid sequence of any one of SEQ ID NOs: 1 to 4 has a visual function restoring capability or visual function deterioration preventing capability. As used herein, “base sequence has a visual function restoring capability or visual function deterioration preventing capability means that a polypeptide encoded by the base sequence has a visual function restoring capability or visual function deterioration preventing capability. A DNA having a base sequence encoding the amino acid sequence of any one of SEQ ID NOs: 1 to 4 further encompasses various mutants and homologs having a visual function restoring capability or visual function deterioration preventing capability. Mutants and homologs of a DNA having a base sequence encoding the amino acid sequence of any one of SEQ ID NOs: 1 to 4 encompass, for example, DNAs having a base sequence that can hybridize under a stringent condition with a base sequence encoding the amino acid sequence of any one of SEQ ID NOs: 1 to 4. Further, mutants and homologs of a DNA having a base sequence encoding the amino acid sequence of any one of SEQ ID NOs: 5 to 7 encompass DNAs having a base sequence that can hybridize under a stringent condition with a base sequence encoding the amino acid sequence of any one of SEQ ID NOs: 5 to 7. Examples of “stringent condition” include conditions for performing a reaction at 40 to 70° C. (preferably 50 to 67° C. and more preferably 60 to 65° C.) in a normal hybridization buffer and washing in a detergent with a salt concentration of 15 to 300 mM (preferably 15 to 150 mM, more preferably 15 to 60 mM, and still more preferably 30 to 50 mM).

Any one of SEQ ID NOs: 1 to 4 can be used as the amino acid sequence of the chimeric protein of the invention. A DNA encoding the amino acid sequence of the chimeric protein of the invention encompasses DNAs having a base sequence encoding the amino acid sequence of any one of SEQ ID NOs: 1 to 4 with one or more amino acid substitutions, deletions, and/or additions. In this regard, “one or more” in any one of SEQ ID NOs: 1 to 4 is generally 50 amino acids or less, preferably 30 amino acids or less, and still more preferably 10 amino acids or less (e.g., 5 amino acids or less, 3 amino acids or less, or one amino acid). Further, “one or more” in any one of SEQ ID NOs: 5 to 7 is generally 6 amino acids or less, preferably 5 amino acids or less, and still more preferably 4 amino acids or less (e.g., 3 amino acids or less, 2 amino acids or less, and one amino acid). When maintaining a visual function restoring capability or visual function deterioration preventing capability of a chimeric protein, it is desirable that an amino acid residue to be mutated is mutated to another amino acid which conserves the property of an amino acid side chain. Examples of properties of an amino acid side chain include hydrophobic amino acids (A, I, L, M, F, P, W, Y, V), hydrophilic amino acids (R, D, N, C, E, Q, G, H, K, S, T), amino acids with an aliphatic side chain (G, A, V, L, I, P), amino acids with a hydroxyl group containing side chain (S, T, Y), amino acids with a sulfur atom containing side chain (C, M), amino acids with a carboxylic acid and amide containing side chain (D, N, E, Q), amino acids with a base containing side chain (R, K, H), and amino acids with an aromatic containing side chain (H, F, Y, W) (each symbol within the parenthesis represents the one-letter code of an amino acid). It is known that proteins having an amino acid sequence modified by one or more amino acid residue deletions, additions, and/or substitutions with another amino acid to the amino acid sequence maintain the biological activity thereof (Mark, D. F. et al., Proc. Natl. Acad. Sci. USA (1984) 81, 5662-5666, Zoller, M. J.& Smith, M. Nucleic Acids Research (1982) 10, 6487-6500, Wang, A. et al., Science 224, 1431-1433, Dalbadie-McFarland, G. et al., Proc. Natl. Acad. Sci. USA (1982) 79, 6409-6413).

Mutants and homologs of a DNA having a base sequence encoding the amino acid sequence of any one of SEQ ID NOs: 1 to 4 encompass DNAs consisting of a base sequence having high homology with a base sequence encoding the amino acid sequence of any one of SEQ ID NOs: 1 to 4. Such a DNA preferably has homology of 90% or greater, and still more preferably 95% or greater (96% or greater, 97% or greater, 98% or greater, or 99% or greater) with a base sequence encoding the amino acid sequence of any one of SEQ ID NOs: 1 to 4. Mutants and homologs of a DNA having a base sequence encoding the amino acid sequence of any one of SEQ ID NOs: 5 to 7 encompass DNAs consisting of a base sequence having high homology with a base sequence encoding the amino acid sequence of any one of SEQ ID NOs: 5 to 7. Such a DNA preferably has homology of 90% or greater, and still more preferably 95% or greater (96% or greater, 97% or greater, 98% or greater, or 99% or greater) with a base sequence encoding the amino acid sequence of any one of SEQ ID NOs: 5 to 7. The homology of amino acid sequences and base sequences can be determined by the algorithm BLAST developed by Karlin and Altschul (Proc. Natl. Acad. Sci. USA 90: 5873-5877, 1993). Programs called BLASTN and BLASTX have been developed based on this algorithm (Altschul et al. J. Mol. Biol. 215: 403-410, 1990). When analyzing a base sequence using BLASTN based on BLAST, parameters are set to, for example, score=100 and wordlength=12. When analyzing an amino acid sequence using BLASTX based on BLAST, parameters are set to, for example, score=50 and wordlength=3. When using BLAST and Gapped BLAST programs, the default parameters of each program are used. The specific approaches of these analysis methods are known (http://www.ncbi.nlm.nih.gov).

As used herein, “DNA” may be a sense strand or an antisense strand (e.g., can be used as a probe). The shape thereof may be a single strand or double strand. DNA may also be genomic DNA, cDNA, or synthesized DNA.

The method of obtaining DNA of the invention is not particularly limited. Examples thereof include known methods such as a method of obtaining cDNA by reverse transcription from mRNA (e.g., RT-PCR method), method of adjusting from genomic DNA, method of synthesizing by chemical synthesis, and method of isolating from a genomic DNA library or a cDNA library (see, for example, Japanese Laid-Open Publication No. 11-29599).

A chimeric protein used in the agent for restoring visual function or agent for preventing deterioration in visual function of the invention can be prepared, for example, by using a transformant introduced with an expression vector comprising a DNA encoding the aforementioned chimeric protein. For example, the transformant is first cultured under suitable conditions to synthesize a chimeric protein encoded by the DNA. The synthesized protein can then be retrieved from the transformant or culture to obtain the chimeric protein of the invention.

More specifically, this can be made by inserting a DNA encoding the aforementioned chimeric protein into a suitable expression vector. The “suitable vector” may be any vector that can be replicated and retained or self-proliferate within various hosts of prokaryotes and/or eukaryotes. The vector can be appropriately selected depending on the objects of use. For obtaining a large quantity of DNA, a high copy number vector, for example, can be selected. For obtaining a polypeptide (chimeric protein), an expression vector can be selected. Specific examples of vectors include, but are not particularly limited to, known vectors described in Japanese Laid-Open Publication No. 11-29599.

Expression vectors can not only synthesize a chimeric protein, but also be used in the agent for restoring visual function or agent for preventing deterioration in visual function of the invention. In other words, the agent for restoring visual function or agent for preventing deterioration in visual function of the invention may comprise, as an active ingredient, an expression vector into which a DNA encoding the amino acid sequence of the aforementioned chimeric protein is incorporated. Such an expression vector can be used in restoring visual function or prevention of deterioration in visual function by direct introduction into a human. As a vector in such use, a vector that can be introduced into a human cell is used. Preferred examples of such a vector include adeno-associated virus vectors (AAV vectors) and lentivirus vectors.

A method of introducing a vector can be appropriately selected depending on the type of host or vector or the like. Specific examples of the method include, but are not particularly limited to, known methods such as the protoplast and competent methods when bacteria are used as the host (see, for example, Japanese Laid-Open Publication No. 11-29599). When an expression vector is used as an active ingredient of the agent for restoring visual function or agent for preventing deterioration in visual function of the invention, the aforementioned AAV vector or the like can be introduced, for example, by injection into the eye.

A host to which an expression vector may be any host that is compatible with the expression vector and can be transformed. Specific examples of the host include, but are not particularly limited to, known naturally-occurring or artificially established cells such as bacteria, yeast, animal cells, and insect cells (see Japanese Laid-Open Publication No. 11-29599) and animals such as humans and mice. A transformant can be cultured by suitably selecting a medium from known nutrient media depending on the type of the transformant or the like and appropriately adjusting the temperature, pH of the nutrient medium, culture time, and the like, so that a chimeric protein can be readily obtained in large quantities (see, for example, Japanese Laid-Open Publication No. 11-29599).

An isolation method and purification method of a chimeric protein is not particularly limited. Examples thereof include known methods such as methods of utilizing solubility, methods utilizing the difference in molecular weights, and methods utilizing charges (see, for example, Japanese Laid-Open Publication No. 11-29599).

As used herein, “active ingredient” refers to an ingredient contained at an amount needed to attain the effect of restoring visual function or the effect of preventing deterioration in visual function. Other ingredients may also be contained, as long as the effect is not reduced below a desired level. The agent for restoring visual function or agent for preventing deterioration in visual function of the invention may also be formulated. Further, the route of administration of the agent for restoring visual function or agent for preventing deterioration in visual function of the invention may be either oral or parenteral. The route of administration can be appropriately determined depending on the form of formulation or the like.

For oral administration, the agent may be formulated into various forms such as tablets, granules, fine granules, powder, and capsules for use. An additive commonly used in a formulation such as a binding agent, covering agent, excipient, lubricant, disintegrant, or humectant may also be included. In addition thereto, formulations for oral administration may be formulated as a liquid formulation such as an aqueous solution for internal use, suspension, emulsion, or syrup. The formulation may also be formulated as a dry formulation that is dissolved in a solvent upon use.

For parenteral administration, the agent may be formulated to be contained in a unit dose ampule or multidose container or tube. An additive such as a stabilizer, buffer, preservative, or isotonizing agent may also be included. A formulation for parenteral administration may also be formulated into a powder form that can be dissolved in a suitable carrier (sterilized water or the like) upon use.

Examples of parenteral administration include intravitreal administration, subconjunctival administration, intra-anterior chamber administration, and eye drops, and intravitreal administration is preferred.

The agent for restoring visual function or agent for preventing deterioration in visual function of the invention discussed above can be used for restoring visual function or preventing deterioration in visual function by administration to humans using the aforementioned method.

As use herein, “visual function restoration” refers to improvement of deteriorated visual function, which may be a partial or complete restoration of the visual function. Further, “prevention of deterioration in visual function” refers to prevention of deterioration in visual function, suppression of progression in deterioration of visual function, and the like. Examples of such visual function include vision, contrast sensitivity, light adaptation, color perception, and the like.

The agent for restoring visual function or agent for preventing deterioration in visual function of the invention may be used in applications expected from restoration of visual function or prevention of deterioration in visual function. For example, the agent may be used in treating or preventing a disease associated with deterioration in visual function. Examples of diseases associated with deterioration in visual function include retinitis pigmentosa, age related macular degeneration, myopic maculopathy, macular dystrophy, diabetic retinopathy, uveitis, retinal detachment, and the like.

### <Vector>

The present invention includes adeno-associated virus (AAV) vectors and lentivirus vectors, to which a sequence of a chimeric protein having an amino acid sequence of a microorganism derived ion transport receptor rhodopsin and an amino acid sequence of an animal derived G protein-coupled receptor rhodopsin is inserted.

The present invention also includes the use of an adeno-associated virus (AAV) vector or lentivirus vector, to which a sequence of a chimeric protein having an amino acid sequence of a microorganism derived ion transport receptor rhodopsin and an amino acid sequence of an animal derived G protein-coupled receptor rhodopsin is inserted, for the manufacture of a medicament for restoring visual function or preventing deterioration in visual function.

The same chimeric protein discussed above can be used as the chimeric protein.

### Examples

Experiments related to visual function were conducted using mice as described below.

### (Experimental Animal)

For the experiments, wild-type mouse (C57BL/6J, CLEA Japan Inc.) and retinitis pigmentosa model (rd1) mouse (C3H/HeJ Jcls, CLEA Japan Inc.) were used, which were both 3-week old male.

### (Production of DNA Encoding Chimeric Protein (GR/BvRh)

A DNA encoding a chimeric protein, in which a sequence corresponding to 137th to 145th amino acids from the N-terminus corresponding to the second loop on the cytoplasm side of Gloeobacter violaceus Rhodopsin ((GR), SEQ ID NO: 8) was replaced with a sequence corresponding to 137th to 145th amino acids of a bovine rhodopsin (BvRh) (SEQ ID NO: 9), and a sequence corresponding to 198th to 206th amino acids from the N-terminus corresponding to the third loop on the cytoplasm side of Gloeobacter violaceus Rhodopsin was replaced with a sequence corresponding to 225th to 252th amino acids of the bovine rhodopsin, and the 132nd amino acid, glutamic acid, of Gloeobacter violaceus Rhodopsin was replaced with glutamine, was inserted into a pCDNA3.1 vector. The mutant was produced by the QuicChange method.

(Production of Adeno-Associated Virus (AAV) Vector to which a Sequence of Chimeric Protein is Inserted)

An EGFP or GR/BvRh gene was subcloned to an AAV2 shuttle plasmid to produce AAV2-CAGGS-EGFP-WPRE-pA (vector for expressing EGFP) and AAV2-CAGGS-GR/BvRh-WPRE-pA (vector for expressing a chimeric protein) as virus expressing constructs. Viral vectors were packaged by transfection of three types of plasmids, i.e., vector plasmid, AAV vector plasmid, and adenovirus helper plasmid, into HEK 293 cells. Cesium chloride method was used for the purification of the viral vectors. In the vectors, “ITR” is an abbreviation for “Inverted Terminal Repeat”. “CAGGS” is a sequence of a region of a CAG promoter. “WPRE” is an abbreviation for “woodchuck hepatitis virus post-transcriptional regulatory element”. “pA” refers to a peptide tag. “EGFP” is an abbreviation for “enhanced green fluorescent protein”.

### (Vitreous Body Injection)

A mixture of medetomidine hydrochloride (0.75 mg/kg), midazolam (4 mg/kg), butorphanol tartrate (5 mg/kg) was intraperitoneally administered to a wild-type mouse or retinitis pigmentosa model (rd1) mouse. Under systemic anesthesia, a microsyringe equipped with a 32 gauge needle was used to inject the aforementioned AAV vector (“AAV2-CAGGS-EGFP-WPRE-pA” or “AAV2-CAGGS-GR/BvRh-WPRE-pA”) at 1×10¹² vg/ml and 1 μl, respectively, into the vitreous body from near the ora serrata.

### (Reporter Observation)

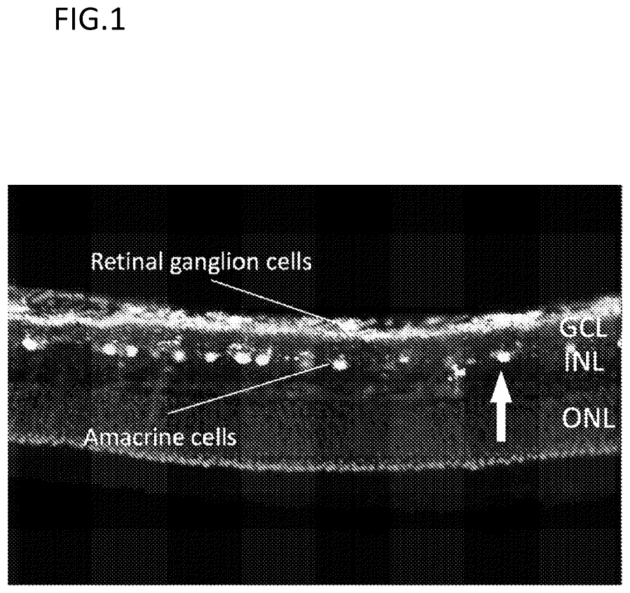

The retina was extracted from a wild-type mouse injected with AAV2-CAGGS-EGFP-WPRE-pA after 7 weeks from injection and immobilized for 1 hour with 4% paraformaldehyde. The whole-mounted retina was observed under a fluorescence microscope. FIG. 1 shows the result thereof. In FIG. 1, GCL means the ganglion cell layer, INL means the inner nuclear layer, and ONL means the outer nuclear layer. Green fluorescence (e.g., arrow in FIG. 1) was observed in the retina as a result of observation. Thus, it was possible to confirm that vector introduction and expression of a gene of interest were normal.

### (Multielectrode Array Recording (MEA))

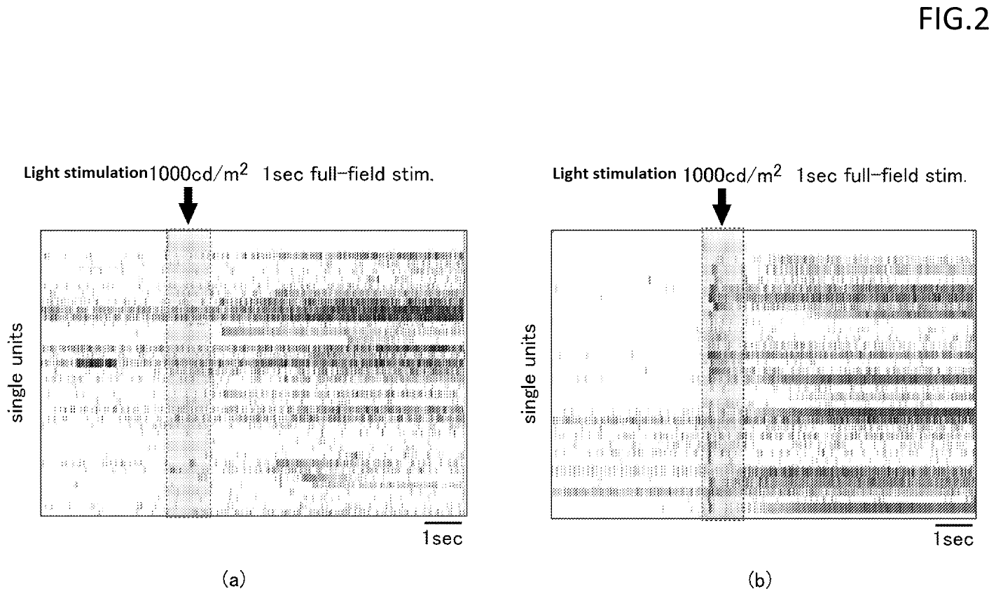

An eye ball was extracted under general anesthesia after 7 weeks from injecting AAV2-CAGGS-GR/BvRh-WPRE-pA to a retinitis pigmentosa model (rd1) mouse. The eye ball was then left standing in an Ames medium (Sigma-Aldrich, St Louis, MO; A1420) bubbled with 95% 02 and 5% CO₂, then the retina was extracted. The retina was mounted so that the ganglion cell layer contacted an electrode facing down, and subjected to light stimulation (white light, 1000 cd/m², 1 second) to record extracellular potential of retinal ganglion cells. Extracellular potential of retinal ganglion cells was also recorded by the same method using a retinitis pigmentosa model (rd1) mouse which had not been injected with AAV2-CAGGS-GR/BvRh-WPRE-pA as a control. A MEA2100-Lite system (Multi-Channel Systems, Reutlingen, Germany) was used for the multielectrode array recording. FIG. 2 shows the results thereof. FIG. 2(a) shows a graph for the control mouse, and FIG. 2(b) shows a graph for a mouse injected with AAV2-CAGGS-GR/BvRh-WPRE-pA. In the graphs of FIG. 2, the horizontal axis indicates the time elapsed, and the regions indicated by an arrow indicate regions where light stimulation was applied.

As shown in FIG. 2, no change was observed in the region where light stimulation was applied for the control, but it was found that the potential increased for the mouse injected with AAV2-CAGGS-GR/BvRh-WPRE-pA. In view of these results, it was found that GR/BvRh has an effect of restoring visual function against retinitis pigmentosa.

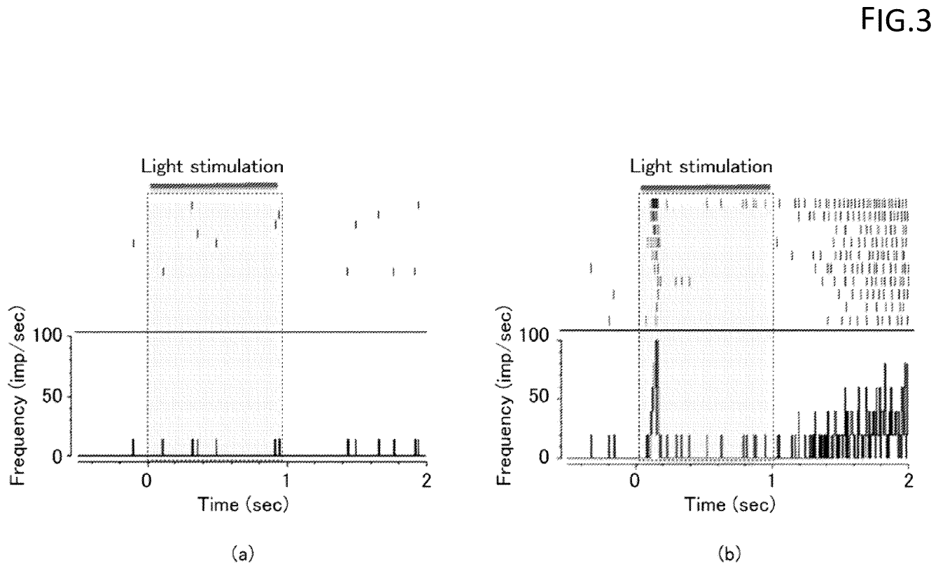

Further, multielectrode array recording was performed by the same approach as above to obtain 10 firings of retinal ganglion cells displayed in a raster plot (top row of FIG. 3), and histograms representing the frequency of firings per second on the vertical axis (bottom row of FIG. 3). Light stimulation was applied from 0 to 1 second. FIG. 3(a) shows a graph for a control mouse, and FIG. 3(b) shows a graph for a mouse injected with AAV2-CAGGS-GR/BvRh-WPRE-pA.

As shown in FIG. 3, a photoresponse was not observed in the control mouse, whereas firing of ganglion cells was observed in the mouse injected with AAV2-CAGGS-GR/BvRh-WPRE-pA, so that a visual restoration effect was observed.

1. An agent for restoring visual function or agent for preventing deterioration in visual function comprising, as an active ingredient, a chimeric protein having an amino acid sequence of a microorganism derived ion transport receptor rhodopsin and an amino acid sequence of an animal derived G protein-coupled receptor rhodopsin.

2. The agent for restoring visual function or agent for preventing deterioration in visual function of claim 1, wherein the chimeric protein has an amino acid sequence of a second loop on a cytoplasm side and/or a third loop on a cytoplasm side of the amino acid sequence of the microorganism derived ion transport receptor rhodopsin substituted with an amino acid sequence of a second loop on a cytoplasm side and/or a third loop on a cytoplasm side of the G protein-coupled receptor rhodopsin.

3. The agent for restoring visual function or agent for preventing deterioration in visual function of claim 1 or 2, wherein the microorganism derived ion transport receptor rhodopsin is a rhodopsin derived from a microorganism of the Gloeobacter genus, and the G protein-coupled receptor rhodopsin is a bovine or human derived rhodopsin.

4. The agent for restoring visual function or agent for preventing deterioration in visual function of any one of claims 1 to 3, wherein the chimeric protein has an amino acid sequence encoded by a DNA of any one of the following (a) to (d):
(a) a DNA having a base sequence encoding the amino acid sequence of any one of SEQ ID NOs: 1 to 4;
(b) a DNA having a base sequence that can hybridize under a stringent condition with a base sequence complementary to a base sequence encoding the amino acid sequence of any one of SEQ ID NOs: 1 to 4;
(c) a DNA having a base sequence encoding the amino acid sequence of any one of SEQ ID NOs: 1 to 4 with one or more amino acid substitutions, deletions, and/or additions, and having a visual function restoring capability or visual function deterioration preventing capability; and
(d) a DNA consisting of a base sequence encoding an amino acid sequence having 90% or greater homology with the amino acid sequence of any one of SEQ ID NOs: 1 to 4 and having a visual function restoring capability or visual function deterioration preventing capability.

5. An agent for restoring visual function or agent for preventing deterioration in visual function comprising, as an active ingredient, an expression vector into which a DNA encoding the amino acid sequence of the chimeric protein of any one of claims 1 to 4 is incorporated.

6. The agent for restoring visual function or agent for preventing deterioration in visual function of any one of claims 1 to 5 for use in treating or preventing retinitis pigmentosa.

7. An adeno-associated virus (AAV) vector or lentivirus vector, to which a sequence of a chimeric protein having an amino acid sequence of a microorganism derived ion transport receptor rhodopsin and an amino acid sequence of an animal derived G protein-coupled receptor rhodopsin is inserted.

8. Use of an adeno-associated virus (AAV) vector or lentivirus vector, to which a sequence of a chimeric protein having an amino acid sequence of a microorganism derived ion transport receptor rhodopsin and an amino acid sequence of an animal derived G protein-coupled receptor rhodopsin is inserted, for the manufacture of a medicament for restoring visual function or preventing deterioration in visual function.


SEQUENCE LISTING (9 sequences)

SEQ ID NO: 1 - protein, 317 aa, synthetic construct
  REGION 1..317: note = Artificial Peptide
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVQTVAVIERYVVVCKPMSNFRFGENHPLLIKLTVASVLMIATGYPGEISDDITTRIIWGTVSTIPFAYILYVLWVELSFTVKEAAAQQQESATTQVQTLVRNMRWLLLLSWGVYPIAYLLPMLGVSGTSAAVGVQVGYTIADVLAKPVFGLLVFAIALVKTKADQESSEPHAAIGAAANKSGGSLIS

SEQ ID NO: 2 - protein, 344 aa, synthetic construct
  REGION 1..344: note = Artificial Peptide
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVQTVAVIERYVVVCKPMSNFRFGENHPLLIKLTVASVLMIATGYPGEISDDITTRIIWGTVSTIPFAYILYVLWVELSFTVKEAAAQQQESATTQVQTLVRNMRWLLLLSWGVYPIAYLLPMLGVSGTSAAVGVQVGYTIADVLAKPVFGLLVFAIALVKTKADQESSEPHAAIGAAANKSGGSLISGSASASNGASDYKDDDDKEFFCYENEV

SEQ ID NO: 3 - protein, 344 aa, synthetic construct
  REGION 1..344: note = Artificial Peptide
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVETVAVIERYVVVCKPMSNFRFGENHPLLIKLTVASVLMIATGYPGEISDDITTRIIWGTVSTIPFAYILYVLWVELSFTVKEAAAQQQESATTQVQTLVRNMRWLLLLSWGVYPIAYLLPMLGVSGTSAAVGVQVGYTIADVLAKPVFGLLVFAIALVKTKADQESSEPHAAIGAAANKSGGSLISGSASASNGASDYKDDDDKEFFCYENEV

SEQ ID NO: 4 - protein, 311 aa, synthetic construct
  REGION 1..311: note = Artificial Peptide
MASQVVYGEWASTHTECYNMSRIDSTFVSLLQLVWAVVSGCQTIFMISRAPKVPWESVYLPFVESITYALASTGNGTLQMRDGRFFPWSRMASWLCTCPIMLGQISNMIERYVVVCKPMSNFRFGENHIPLNPIAQAASIIRVVMGITATISPAEYMKWLFFFFGATCLVFEYSVVFTIFQVGLFTVKEAAAQQQESATTQAQKVVVRIKMLRLIFFIAWTMFPIVWLISPTGVCVIHENVSAILYLLADGLCKNTYGVILWSTAWGVLEGKWDPACLPGQEKPEADDPFGLNHEKNAPPNDEVNIRMFGR

SEQ ID NO: 5 - protein, 11 aa, synthetic construct
  REGION 1..11: note = Artificial Peptide
IERYVVVCKPM

SEQ ID NO: 6 - protein, 20 aa, synthetic construct
  REGION 1..20: note = Artificial Peptide
IERYVVVCKPMSNFRFGENH

SEQ ID NO: 7 - protein, 17 aa, synthetic construct
  REGION 1..17: note = Artificial Peptide
FTVKEAAAQQQESATTQ

SEQ ID NO: 8 - protein, 298 aa, Gloeobacter violaceus
MLMTVFSSAPELALLGSTFAQVDPSNLSVSDSLTYGQFNLVYNAFSFAIAAMFASALFFFSAQALVGQRYRLALLVSAIVVSIAGYHYFRIFNSWDAAYVLENGVYSLTSEKFNDAYRYVDWLLTVPLLLVETVAVLTLPAKEARPLLIKLTVASVLMIATGYPGEISDDITTRIIWGTVSTIPFAYILYVLWVELSRSLVRQPAAVQTLVRNMRWLLLLSWGVYPIAYLLPMLGVSGTSAAVGVQVGYTIADVLAKPVFGLLVFAIALVKTKADQESSEPHAAIGAAANKSGGSLIS

SEQ ID NO: 9 - protein, 348 aa, Bos taurus
MNGTEGPNFYVPFSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVAFTWVMALACAAPPLVGWSRYIPEGMQCSCGIDYYTPHEETNNESFVIYMFVVHFIIPLIVIFFCYGQLVFTVKEAAAQQQESATTQKAEKEVTRMVIIMVIAFLICWLPYAGVAFYIFTHQGSDFGPIFMTIPAFFAKTSAVYNPVIYIMMNKQFRNCMVTTLCCGKNPLGDDEASTTVSKTETSQVAPA